(19) United States
(12) Patent Application Publication
(10) Pub. No.: US 2023/0293301 A1
(43) Pub. Date: Sep. 21, 2023

(54) FLUID CONTROL SYSTEM FOR AN IMPLANTABLE INFLATABLE DEVICE

(71) Applicant: Boston Scientific Scimed, Inc., Maple Grove, MN (US)

(72) Inventors: Thomas Sinnott, Enniscorthy (IE); Daragh Nolan, Via Youghal (IE); Noel Smith, Windgap (IE); Eduardo Marcos Larangeira, Cork City (IE); Evania Ann Mareena, Clonmel (IE); Sean Cooney, Meelick (IE)

(21) Appl. No.: 18/182,622

(22) Filed: Mar. 13, 2023

Related U.S. Application Data
(60) Provisional application No. 63/269,438, filed on Mar. 16, 2022.

(51) Int. Cl. A61F 2/26; A61F 2/48

(52) U.S. Cl. CPC A61F 2/26; A61F 2/482; A61F 2/484; A61F 2250/0013

(57) ABSTRACT

An implantable fluid operated device may include a fluid reservoir configured to hold fluid, an inflatable member, and an electronic fluid control system to transfer fluid between the fluid reservoir and the inflatable member. The fluid control system includes at least one pump or at least one valve including a piezoelectric actuator. The piezoelectric actuator can include a piezoelectric element that deforms in response to a voltage applied by an electronic control system of the fluid control system, and a diaphragm that deforms in response to deformation of the piezoelectric element. An isolation layer may be coupled to the piezoelectric element to isolate the active piezoelectric element from non-active portions of the piezoelectric actuator.

### CROSS-REFERENCE TO RELATED APPLICATION

This application claims priority to U.S. Provisional Pat. Application No. 63/269,438, filed on Mar. 16, 2022, entitled “FLUID CONTROL SYSTEM FOR AN IMPLANTABLE INFLATABLE DEVICE”, the disclosure of which is incorporated by reference herein in its entirety.

### TECHNICAL FIELD

This disclosure relates generally to bodily implants, and more specifically to bodily implants including a fluid control system having one or more pumps and/or valves including a piezoelectric actuator.

### BACKGROUND

Active implantable fluid operated inflatable devices often include one or more pumps that regulate a flow of fluid between different portions of the implantable device. One or more valves can be positioned within fluid passageways of the device to direct and control the flow of fluid to achieve inflation, deflation, pressurization, depressurization, activation, deactivation and the like of different fluid filled implant components of the device. In some implantable fluid operated devices, an implantable pumping device may be manually operated by the user to provide for the transfer of fluid between a reservoir and the fluid filled implant components of the device. Manipulation of the manually operated implantable pumping device may be challenging for some patients. Further, such manual operation of the pumping device may make it may be difficult to achieve consistent inflation, deflation, pressurization, depressurization, activation, deactivation and the like of the fluid filled implant components. Inconsistent inflation, deflation, pressurization, depressurization, activation and/or deactivation of the fluid filled implant device(s) may adversely affect patient comfort, efficacy of the device, and the overall patient experience. Accurate actuation and control of a fluid control system controlling the flow of fluid between components of the inflatable device will improve performance and efficacy of the device, and will improve patient comfort and safety

### SUMMARY

In a general aspect, an implantable fluid operated inflatable device includes a fluid reservoir, an inflatable member, and a fluid control system configured to control fluid flow between the fluid reservoir and the inflatable member. In some examples, the fluid control system includes a housing, fluidic architecture defining one or more fluid passageways within the housing, and a piezoelectric actuator actuating at least one pump or at least one valve positioned in the one or more fluid passageways. In some examples, the piezoelectric actuator includes a deformable member mounted in a fluid passageway of the one or more fluid passageways defined within the housing to control a flow of fluid through the fluid passageway, a piezoelectric element coupled to the deformable member and configured to deform in response to a voltage applied by an electronic control system of the fluid control system, and an isolation layer positioned between the piezoelectric element and the deformable member and configured to electrically isolate the deformable member from the piezoelectric element.

In some implementations, the implantable fluid operated inflatable device includes a first epoxy layer coupling the isolation layer to the piezoelectric element, and a second epoxy layer coupling the isolation layer to the deformable member. In some implementations, a material of the isolation layer is a dielectric material, and a material of at least one of the first epoxy layer or the second epoxy layer is a polymer material processed prior to application to remove voids. In some implementations, an outer peripheral dimension of the isolation layer is greater than or equal to an outer peripheral dimension of the piezoelectric element. In some implementations, the isolation layer includes a mesh material or a woven material having a set thickness across the isolation layer.

In some implementations, the piezoelectric element includes at least one electrode on a first side of the piezoelectric element, and at least one recess formed in a second side of the piezoelectric element, at a position corresponding to the at least one electrode. In some implementations, the isolation layer includes an epoxy layer, and wherein a thickness of the epoxy later at a position corresponding to the at least one recess and the at least one electrode is greater than a thickness of remaining portions of the epoxy layer.

In some implementations, the piezoelectric element includes a first cutaway portion corresponding to a placement position of a first electrode relative to the piezoelectric element, and a second cutaway portion corresponding to a placement position of a second electrode relative to the piezoelectric element. In some implementations, a thickness of a portion of the isolation layer corresponding to the first cutaway portion, and a thickness of a portion of the isolation layer corresponding to the second cutaway portion, is greater than a thickness of remaining portions of the isolation layer. In some implementations, the isolation layer includes an epoxy layer, and wherein a material stiffness of the epoxy layer corresponds to a material stiffness of the piezoelectric element.

In another general aspect, an implantable fluid operated inflatable device includes a fluid reservoir, an inflatable member, and a fluid control system coupled between the fluid reservoir and the inflatable member and configured to control fluid flow between the fluid reservoir and the inflatable member. In some implementations, the fluid control system includes a housing, fluidic architecture defining one or more fluid passageways within in the housing, and a piezoelectric actuator actuating at least one pump and at least one valve positioned in the one or more fluid passageways. In some implementations, the piezoelectric actuator includes a piezoelectric element configured to deform in response to a voltage applied by an electronic control system of the fluid control system, an actuator foil coupled to the piezoelectric element, and an isolation layer between the piezoelectric element and the actuator foil.

In some implementations, the isolation layer includes a coating layer deposited on one of the actuator foil or the piezoelectric element, the coating layer including a nano-thickness layer of a ceramic material deposited on the one of the actuator foil or the piezoelectric element, and an epoxy layer between the coating layer and the other of the actuator foil or the piezoelectric element. In some implementations, the isolation layer includes at least one ceramic layer, a first epoxy layer bonding the at least one ceramic layer and the piezoelectric element, and a second epoxy layer bonding the at least one ceramic layer and the actuator foil. In some implementations, the isolation layer includes a plurality of microbeads positioned between the piezoelectric element and the actuator foil, wherein the plurality of microbeads are made of an insulative material and have a set size so as to maintain a set distance between the piezoelectric element and the actuator foil, and an epoxy material applied between the piezoelectric element and the actuator foil and configured to bond the piezoelectric element, the actuator foil, and the plurality of microbeads. In some implementations, the isolation layer includes a mesh material positioned between the piezoelectric element and the actuator foil, wherein the mesh material is made of an insulative material and has a set thickness so as to maintain a set distance between the piezoelectric element and the actuator foil, and an epoxy material applied between the piezoelectric element and the actuator foil, and in openings in the mesh material, and configured to bond the piezoelectric element, the actuator foil, and the mesh material.

In another general aspect, an implantable fluid operated inflatable device includes a fluid reservoir, an inflatable member, and a fluid control system configured to control fluid flow between the fluid reservoir and the inflatable member. In some implementations, the fluid control system includes a housing, fluidic architecture defining one or more fluid passageways within the housing, and a piezoelectric actuator actuating at least one pump and at least one valve positioned in the one or more fluid passageways. In some implementations, the piezoelectric actuator includes a deformable member mounted in a fluid passageway of the one or more fluid passageways defined within the housing to control a flow of fluid through the fluid passageway, a piezoelectric element coupled to the deformable member and configured to deform in response to a voltage applied by an electronic control system of the fluid control system, and an isolation layer positioned between the piezoelectric element and the deformable member and configured to electrically isolate the deformable member from the piezoelectric element.

In some implementations, the piezoelectric actuator includes a first epoxy layer coupling the isolation layer to the piezoelectric element, and a second epoxy layer coupling the isolation layer to the deformable member. In some implementations, at least one of the first epoxy layer or the second epoxy layer is applied in a pattern, the pattern including one of a pattern corresponding to a contour of at least one of the piezoelectric element or the deformable member, a lined pattern, a mesh pattern, or a sawtooth pattern. In some implementations, a material of at least one of the first epoxy layer or the second epoxy layer is a polymer material processed prior to application to remove voids. In some implementations, an outer peripheral dimension of the isolation layer is greater than or equal to an outer peripheral dimension of the piezoelectric element. In some implementations, the isolation layer includes a mesh material or a woven material having a set thickness across the isolation layer. In some implementations, the isolation layer includes a dielectric material.

In some implementations, the piezoelectric element includes at least one electrode on a first side of the piezoelectric element, and at least one recess formed in a second side of the piezoelectric element, at a position corresponding to the at least one electrode. In some implementations, a contour of the at least one recess extends beyond a contour of the at least one electrode. In some implementations, the isolation layer includes an epoxy layer, and wherein a thickness of the epoxy layer at a position corresponding to the at least one recess and the at least one electrode is greater than a thickness of remaining portions of the epoxy layer.

In some implementations, the piezoelectric element includes a first cutaway portion corresponding to a placement position of a first electrode on the piezoelectric element, and a second cutaway portion corresponding to a placement position of a second electrode on the piezoelectric element. In some implementations, a thickness of a portion of the isolation layer corresponding to the first cutaway portion, and a thickness of a portion of the isolation layer corresponding to the second cutaway portion, is greater than a thickness of remaining portions of the isolation layer. In some implementations, the isolation layer includes an epoxy layer, and wherein a material stiffness of the epoxy layer corresponds to a material stiffness of the piezoelectric element.

In another general aspect, an implantable fluid operated inflatable device includes a fluid reservoir, an inflatable member, and a fluid control system coupled between the fluid reservoir and the inflatable member and configured to control fluid flow between the fluid reservoir and the inflatable member. In some implementations, the fluid control system includes a housing, fluidic architecture defining one or more fluid passageways within in the housing, and a piezoelectric actuator actuating at least one pump or at least one valve positioned in the one or more fluid passageways. In some implementations, the piezoelectric actuator includes a piezoelectric element configured to deform in response to a voltage applied by an electronic control system of the fluid control system, an actuator foil coupled to the piezoelectric element, and an isolation layer between the piezoelectric element and the actuator foil.

In some implementations, the isolation layer includes a coating layer deposited on one of the actuator foil or the piezoelectric element, and an epoxy layer between the coating layer and the other of the actuator foil or the piezoelectric element. In some implementations, the coating layer includes a nano-thickness layer of a ceramic material deposited on the one of the actuator foil or the piezoelectric element. In some implementations, the isolation layer includes at least one ceramic layer, a first epoxy layer bonding the at least one ceramic layer and the piezoelectric element, and a second epoxy layer bonding the at least one ceramic layer and the actuator foil. In some implementations, an outer peripheral contour of the at least one ceramic layer is greater than or equal to a corresponding outer peripheral contour of the piezoelectric element. In some implementations, the isolation layer includes a plurality of microbeads positioned between the piezoelectric element and the actuator foil, wherein the plurality of microbeads are made of an insulative material and have a set size so as to maintain a set distance between the piezoelectric element and the actuator foil, and an epoxy material applied between the piezoelectric element and the actuator foil and configured to bond the piezoelectric element, the actuator foil, and the plurality of microbeads. In some implementations, the isolation layer includes a mesh material positioned between the piezoelectric element and the actuator foil, wherein the mesh material is made of an insulative material and has a set thickness so as to maintain a set distance between the piezoelectric element and the actuator foil, and an epoxy material applied between the piezoelectric element and the actuator foil, and in openings in the mesh material, and configured to bond the piezoelectric element, the actuator foil, and the mesh material.

### BRIEF DESCRIPTION OF THE DRAWINGS

FIG. 1 is a block diagram of an implantable fluid operated inflatable device according to an aspect.

FIG. 2A illustrates a first system including a first example implantable fluid operated inflatable device according to an aspect.

FIG. 2B illustrates a second system including a second example implantable fluid operated inflatable device according to an aspect.

FIG. 3A is a schematic view of an example active valve, in an open state.

FIG. 3B is a schematic view of the example active valve shown in FIG. 3A, in a closed state.

FIG. 4 is an exploded view of an example pump actuatable by piezoelectric actuator, according to an aspect.

FIG. 5A is a plan view of an example piezoelectric element, according to an aspect.

FIG. 5B is a perspective view of an example actuator foil.

FIG. 6A schematically illustrates an example piezoelectric element.

FIG. 6B schematically illustrates the coupling of the example piezoelectric element shown in FIG. 6A to a deformable member.

FIG. 6C illustrates the machining of a portion of the example piezoelectric element, according to an aspect.

FIG. 6D schematically illustrates the coupling of the example machined piezoelectric element shown in FIG. 6C to a deformable member according to an aspect.

FIG. 7 schematically illustrates an example piezoelectric element including an insulative coating material, according to an aspect.

FIG. 8A schematically illustrates an example piezoelectric element having an insulative ceramic layer coupled thereto, according to an aspect.

FIG. 8B illustrates an example piezoelectric element having multiple insulative ceramic layers coupled thereto, according to an aspect.

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the implementations in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the implementations are directed to bodily implants. The term patient or user may hereinafter be used for a person who benefits from the medical device or the methods disclosed in the present disclosure. For example, the patient can be a person whose body is implanted with the medical device or the method disclosed for operating the medical device by the present disclosure.

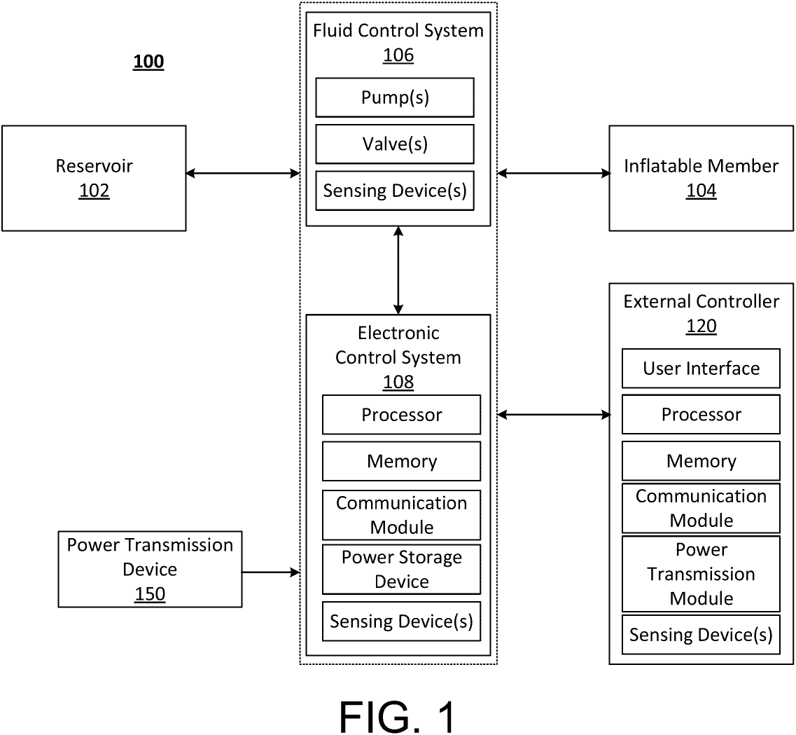

FIG. 1 is a block diagram of an example implantable fluid operated inflatable device 100. The example device 100 shown in FIG. 1 includes a fluid reservoir 102, an inflatable member 104, and a fluid control system 106. The fluid control system 106 can include fluidics components such as one or more pumps, one or more valves and the like configured to transfer fluid between the fluid reservoir 102 and the inflatable member 104. The fluid control system 106 can include one or more sensing devices that sense conditions such as, for example, fluid pressure, fluid flow rate and the like within the fluidics architecture of the inflatable device 100. In some implementations, the inflatable device 100 includes an electronic control system 108. The electronic control system 108 may provide for the monitoring and/or control of the operation of various fluidics components of the fluid control system 106 and/or communication with one or more sensing device(s) within the implantable fluid operated inflatable device 100 and/or communication with one or more external device(s). In some examples, the electronic control system 108 includes components such as a processor, a memory, a communication module, a power storage device, or battery, sensing devices such as, for example an accelerometer, and other such components configured to provide for the operation and control of the implantable fluid operated inflatable device 100. In some examples, the communication module of the electronic control system 108 may provide for communication with one or more external devices such as, for example, an external controller 120.

In some examples, the external controller 120 includes components such as, for example, a user interface, a processor, a memory, a communication module, a power transmission module, and other such components providing for operation and control of the external controller 120 and communication with the electronic control system 108 of the inflatable device 100. For example, the memory may store instructions, applications and the like that are executable by the processor of the external controller 120. The external controller 120 may be configured to receive user inputs via, for example, the user interface, and to transmit the user inputs, for example, via the communication module, to the electronic control system 108 for the processing, operation and control of the inflatable device 100. Similarly, the electronic control system 108 may, via the respective communication modules, transmit operational information to the external controller 120. This may allow operational status of the inflatable device 100 to be provided, for example, through the user interface of the external controller 120, to the user, may allow diagnostics information to be provided to a physician, and the like.

In some examples, the power transmission module of the external controller 120 provides for charging of the components of the internal electronic control system 108. In some examples, transmission of power for the charging of the internal electronic control system 108 can be, alternatively or additionally, provided by an external power transmission device 150 that is separate from the external controller 120. In some implementations the external controller 120 can include sensing devices such as a pressure sensor, an accelerometer, and other such sensing devices. An external pressure sensor in the external controller 120 may provide, for example, a local atmospheric or working pressure to the internal electronic control system 108, to allow the inflatable device 100 to compensate for variations in pressure. An accelerometer in the external controller 120 may provide detected patient movement to the internal electronic control system 108 for control of the inflatable device 100.

The fluid reservoir 102, the inflatable member 104, the fluid control system 106 and the electronic control system 108 may be internally implanted into the body of the patient. In some implementations, the electronic control system 108 is coupled to or incorporated into a housing of the fluid control system 106. In some implementations, at least a portion of the electronic control system 108 is physically separate from the fluid control system 106. In some implementations, some modules of the electronic control system 108 are coupled to or incorporated into the fluid control system 106, and some modules of the electronic control system 108 are separate from the fluid control system 106. For example, in some implementations, some modules of the electronic control system 108 are included in an external device (such as the external controller 120) that is in communication other modules of the electronic control system 108 included within the implantable device 100. In some implementations, at least some aspects of the operation of the implantable fluid operated inflatable device 100 may be manually controlled.

In some examples, electronic monitoring and control of the fluid operated inflatable device 100 may provide for improved patient control of the device, improved patient comfort, and improved patient safety. In some examples, electronic monitoring and control of the fluid operated device 100 may afford the opportunity for tailoring of the operation of the inflatable device 100 by the physician without further surgical intervention. Fluidic architecture defining the flow and control of fluid through the fluid operated inflatable device 100, including the configuration and placement of fluidics components such as pumps, valves, sensing devices and the like, may allow the inflatable device 100 to precisely monitor and control operation of the inflatable device, effectively respond to user inputs, and quickly and effectively adapt to changing conditions both within the inflatable device 100 (changes in pressure, flow rate and the like) and external to the inflatable device 100 (pressure surges due to physical activity, impacts and the like, sustained pressure changes due to changes in atmospheric conditions, and other such changes in external conditions).

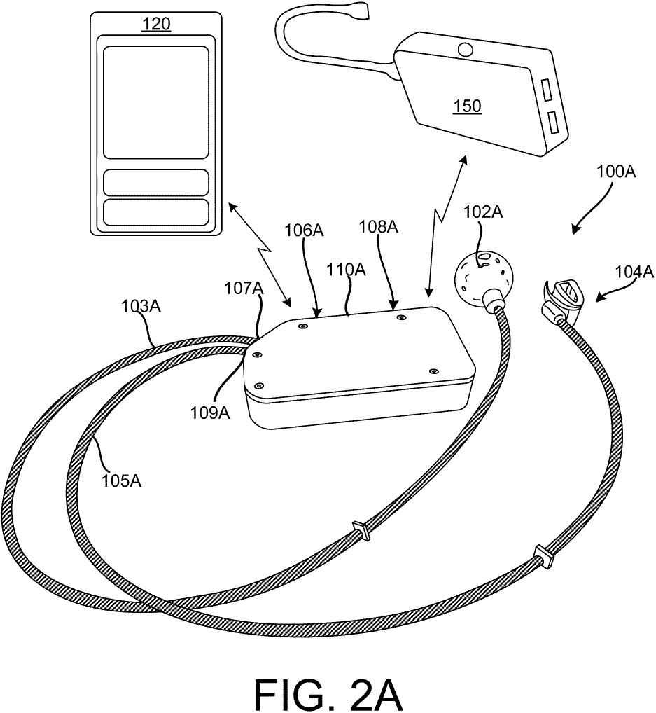

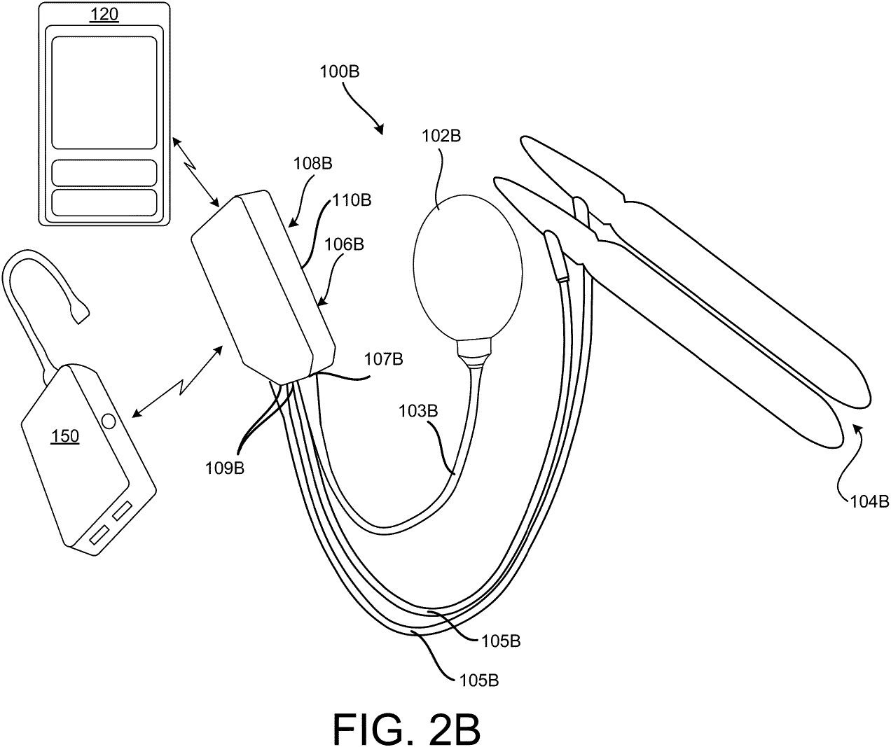

The example implantable fluid operated inflatable device 100 may be representative of a number of different types of implantable fluid operated devices. For example, the device 100 shown in FIG. 1 may be representative of an artificial urinary sphincter 100A as shown in FIG. 2A, an inflatable penile prosthesis 100B as shown in FIG. 2B, and other such implantable inflatable devices that rely on the control of fluid flow to components of the device to achieve inflation, pressurization, deflation, depressurization, deactivation, and the like.

A first example system including a first example implantable fluid operated inflatable device in the form of an example artificial urinary sphincter 100A is shown in FIG. 2A. The artificial urinary sphincter 100A includes a fluid control system 106A including fluidics components such as pumps, valves, sensing devices and the like positioned in fluid passageways, and an electronic control system 108A configured to provide for the transfer of fluid between a reservoir 102A and an inflatable cuff 104A via the fluidics components. Fluidics components of the fluid control system 106A, and electronic components of the electronic control system 108A may be received in a housing 110A. A first conduit 103A connects a first fluid port 107A of the fluid control system 106A/electronic control system 108A received in the housing 110A with the reservoir 102A. A second conduit 105A connects a second fluid port 109A of the fluid control system 106A/electronic control system 108A received in the housing 110A with the inflatable cuff 104A. The electronic control system 108A of the artificial urinary sphincter 100A can communicate with the external controller 120, via the respective communication modules. For example, an application stored in the memory and executed by the processor of the external controller 120 may allow the user and/or a physician to operate, view, monitor and alter operation of the artificial urinary sphincter 100A. In some examples, components of the electronic control system 108A and/or the fluid control system 106A may be charged and/or recharged by a power transmission module of the external controller 120, and/or by a power transmission device 150, that is separate from the external controller 120.

A second example system including a second example implantable fluid operated inflatable device in the form of an example penile prosthesis 100B is shown in FIG. 2B. The penile prosthesis 100B includes a fluid control system 106B including fluidics components such as pumps, valves, sensing devices and the like positioned in fluid passageways, and an electronic control system 108B configured to provide for the transfer of fluid between a fluid reservoir 102B and inflatable cylinders 104B via the fluidics components. Fluidics components of the fluid control system 106B, and electronic components of the electronic control system 108B may be received in a housing 110B. A first conduit 103B connects a first fluid port 107B of the fluid control system 106B/electronic control system 108B received in the housing 110B with the reservoir 102B. One or more second conduits 105B connect one or more second fluid ports 109B of the fluid control system 106A/electronic control system 108A received in the housing with the inflatable cylinders 104B. The electronic control system 108A of the penile prosthesis 100B can communicate with the external controller 120, via the respective communication modules. For example, an application stored in the memory and executed by the processor of the external controller 120 may allow the user and/or a physician to operate, view, monitor and alter operation of the penile prosthesis. In some examples, components of the electronic control system 108A and/or the fluid control system 106A may be charged and/or recharged by a power transmission module of the external controller 120, and/or by a power transmission device 150, that is separate from the external controller 120.

The principles to be described herein may be applied to the example implantable fluid operated inflatable devices shown in FIGS. 2A and 2B, and other types of implantable fluid operated inflatable devices that rely on a pump assembly including various fluidics components to provide for the transfer of fluid between the different fluid filled implantable components to achieve inflation, deflation, pressurization, depressurization, deactivation, occlusion, and the like for effective operation. The example inflatable devices 100A, 100B shown in FIGS. 2A and 2B include electronic control systems 108A, 108B to provide for control of the operation of the respective inflatable members 104A, 104B, and the monitoring and control of pressure and/or fluid flow through the respective inflatable devices 100A, 100B. Some of the principles to be described herein may also be applied to implantable fluid operated inflatable devices that are manually controlled.

As noted above, the fluid control system 106 (106A, 106B) can include a pump assembly including, for example, one or more pumps and one or more valves positioned within a fluid circuit of the pump assembly to control the transfer fluid between the fluid reservoir 102 (102A, 102B) and the inflatable member 104 (104A, 104B). In some examples, the pump(s) and/or the valve(s) are electronically controlled. In some examples, the pump(s) and/or the valve(s) are manually controlled. In an example in which the pump assembly is electronically powered and/or controlled, the pump assembly may include a hermetic manifold that can contain and segment the flow of fluid from electronic components of the pump assembly, to prevent leakage and/or gas exchange. In some examples, the pump(s) and/or valve(s) may include piezoelectric elements. In some examples, the pump assembly includes one or more pressure sensing devices in the fluid circuit to provide for relatively precise monitoring and control of fluid flow and/or fluid pressure within the fluid circuit and/or the inflatable member. A fluid circuit configured in this manner may facilitate the proper inflation, deflation, pressurization, depressurization and deactivation of the components of the implantable fluid operated device to provide for patient safety and device efficacy.

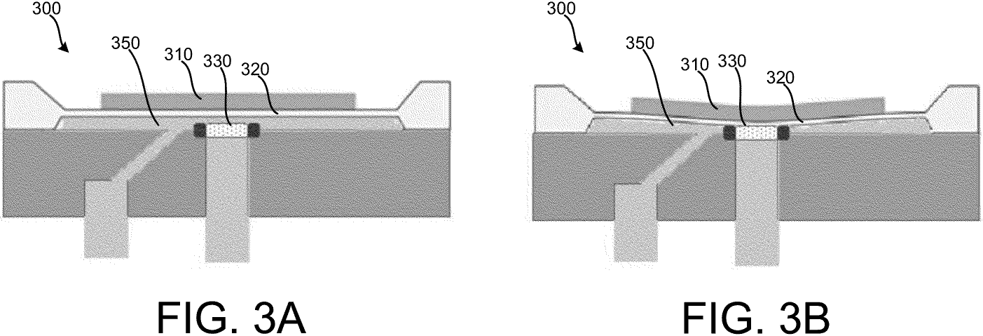

As noted above, in some examples a fluid control system may include one or more pumps and/or one or more valves configured to control the flow of fluid between a reservoir and an inflatable member of an implantable fluid controlled inflatable device, according to an aspect. In some examples, the one or more pumps and/or the one or more valves may include a piezoelectric actuator that provides for relatively precise electronic control of the actuation of the one or more pumps and/or the one or more valves. That is, a piezoelectric actuator may provide for relatively precise control of open and/or closing periods, open and/or closing amounts, fluid flow and flow rates through the fluid passageways of the fluid control system, and the like. FIGS. 3A and 3B schematically illustrate operation of one example of such a valve, including a piezoelectric actuator. The principles to be described herein may be similarly applied to a valve including a piezoelectric actuator.

FIGS. 3A and 3B schematically illustrate the operation and control of a normally open active valve 300 including a piezoelectric element, or a piezoelectric actuator. In particular, FIG. 3A illustrates the open state of the normally open active valve 300, and FIG. 3B illustrates the closed state of the normally open active valve 300. The principles to be described herein may be similarly applied to the operation and control of a normally closed valve including a piezoelectric actuator, the operation and control of a pump including a piezoelectric actuator, the operation of a combination pump and valve including a piezoelectric actuator, and the like.

The example normally open active valve 300 shown in FIGS. 3A and 3B includes a piezoelectric element 310, in the form of a disc made of a piezoelectric material (for example, a piezo-ceramic disc) mounted on a diaphragm 320. In the arrangement shown in FIG. 3A, the normally open active valve 300 is in a default state, or at rest state, or open state, in which fluid can flow through a chamber 350, i.e., from an inlet to an outlet of the chamber 350. In FIG. 3B, the normally open active valve 300 has transitioned to a closed state in response to actuation (for example, application of power to the piezoelectric disc 310). In the arrangement shown in FIG. 3B, the application of power to the piezoelectric disc 310 has caused deformation or deflection of the piezoelectric disc 310, and corresponding deformation or deflection of the diaphragm 320. In the deformed state, the deformed piezoelectric disc 310 and diaphragm 320 press against a sealing element 330 (such as, for example, an O-ring), to close or seal the fluid flow path between the inlet and the outlet of the chamber 350. As noted above, the principles to be described herein may be similarly applied to normally closed valves, and to other types of valves and pumps of the fluidic architecture of an implantable fluid operated inflatable device according to an aspect. In this type of application, the use of a piezoelectric actuator may provide for improved operation and control of the pump and/or valve, improved performance of the implantable fluid operated inflatable device in which the pump and/or valve is installed, and improved patient comfort and safety.

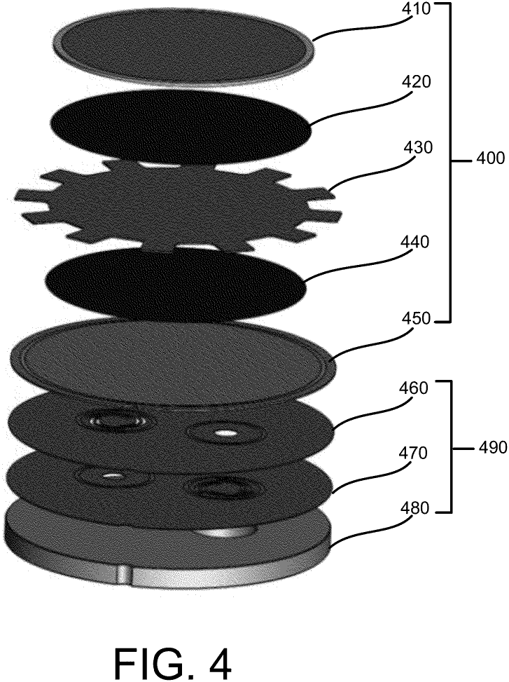

FIG. 4 is an exploded view of an example piezoelectric actuator 400, according to an aspect. FIG. 4 illustrates the use of the example piezoelectric actuator 490 for actuation of a piezoelectric pump 400, simply for purposes of discussion and illustration. As noted above, the principles to be described herein are applicable to other devices, for example, other types of pumps and/or valves and/or combination pump and valves, that are operable with a piezoelectric actuator.

The piezoelectric actuator 400 includes a piezoelectric element 410 that is mounted on a deformable member, such as a diaphragm 450. In the example arrangement shown in FIG. 4, an isolation layer 430 is coupled between the piezoelectric element 410 and the diaphragm 450. A first epoxy layer 420 couples the piezoelectric element 410 and the isolation layer 430, and a second epoxy layer 440 couples the isolation layer 430 and the diaphragm 450. The piezoelectric actuator 400 is operably coupled to the piezoelectric pump 490, including an inlet valve 460 and an outlet valve 470 coupled between the piezoelectric actuator 400 and a base plate 480. In some examples, the piezoelectric actuator can be coupled to device other than the example pump 490 shown in FIG. 4, for actuation of the device.

In the example arrangement shown in FIG. 4, the isolation layer 430 may provide for isolation, for example, electrical isolation, between the piezoelectric element 410 and the diaphragm 450. For example, the isolation layer 430 may maintain a voltage applied to the piezoelectric element 410 within the piezoelectric element 410, and/or may inhibit transmission of voltage applied to the piezoelectric element 410 to areas outside of the piezoelectric element 410. Maintaining the applied voltage within the piezoelectric element 410 and/or inhibiting loss of voltage to areas outside of the piezoelectric element 410 may improve operation and control of the piezoelectric element 410/piezoelectric actuator 400. That is, without any of these types of losses, a known applied voltage may generate a corresponding known magnitude and/or amount and/or direction of bending and/or deformation and/or deflection of the piezoelectric element 410, and a corresponding known magnitude/amount/direction of bending/deformation/deflection of the diaphragm 450. In a situation in which some portion of the known applied voltage is diffused, for example, into adjoining areas, the known applied voltage will produce a different level or magnitude of deformation of the piezoelectric element 410 and the diaphragm 450. Thus, in this situation, the correlation between the known applied voltage and the resulting known deformation of the piezoelectric element 410 and diaphragm 450 is compromised, and operation and control of the piezoelectric actuator 400 and the pump or valve in which it is installed may be adversely impacted. Isolation of voltage to the piezoelectric element 410 may prevent voltage from leaking into fluid in fluid passageways of a pump in which the piezoelectric element is installed, which could otherwise adversely affect the patient, cause corrosion of other components of the pump, cause shorting or other malfunction of the piezoelectric actuator, and the like. Additionally, isolation of voltage to the piezoelectric element 410 inhibits transmission of voltage to the patient, thus enhancing patient safety and comfort. Isolation of the piezoelectric element 410 may prevent voltage from reaching a base plate of the piezoelectric actuator 400 (for example, via the fluid or through direct contact), which could transmit voltage to the housing of the actuator 400 with the possibility for patient contact. Isolation thus improves patient safety and comfort and avoids potential electrochemical interactions with the fluid that could otherwise cause reliability concerns.

Accordingly, the positioning of the isolation layer 430 as shown in FIG. 4 defines a barrier that electrically separates, or isolates, the piezoelectric element 410 and remaining portions of the piezoelectric actuator 400 (for example, the diaphragm 450), despite the physical coupling of the piezoelectric element 410 and the diaphragm 450. The isolation layer may ensure that there is no electrical transfer path from the active piezoelectric element 410 to remaining, non-active portions of the piezoelectric actuator 400, so that substantially all of the (known) voltage applied to the piezoelectric element 410 is manifested in a corresponding known amount, or magnitude of deformation of the piezoelectric element 410, and a corresponding known amount, or magnitude of deformation of the diaphragm 450.

In some examples, the isolation layer 430 may be made of a material having insulative properties, i.e., a non-conductive material, or a material through which current does not flow freely. In some examples, the isolation layer 430 may be made of single or multiple layers of shaped polymetric materials that provide the desired electrical insulative properties, while also being able to bend, or deform, or deflect together with the piezoelectric element 410 and the diaphragm 450, and not impede movement of the piezoelectric element 410 and the diaphragm 450 and/or adversely impact operation of the actuator 400 and the pump or valve in which it is installed. In some examples, the isolation layer 430 may be made of one or more layers of woven materials, that allow the isolation layer 430 to provide the desired electrical insulative properties, while also being able to move with/not impede the movement of the piezoelectric element 410 and the diaphragm 450. Similarly, an overall thickness of the isolation layer 430 may be selected so that the isolation layer 430 provides the desired electrical insulation, while also being able to move with/not impede the movement of the piezoelectric element 410 and the diaphragm 450. In some examples, the isolation layer 430 may include various components such as, for example, an electrode and trace to allow for electrical connection to the piezoelectric element 410.

In some examples, a dimension, for example, an overall dimension, of the isolation layer 430 is greater than a corresponding overall dimension of the piezoelectric element 410. For example, a peripheral portion of the isolation layer 430 may extend beyond a peripheral portion of the piezoelectric element 410. In the example shown in FIG. 4, the piezoelectric element 410 is substantially circular, simply for purposes of discussion and illustration. In this example, an overall diameter of the isolation layer 430 may be greater than or equal to an overall diameter of the piezoelectric element 410. Extension of the outer peripheral portion of the isolation layer 430 beyond the outer peripheral portion of the piezoelectric element 410 ensures that there is no electrical transfer path from the active piezoelectric element 410 to remaining, non-active portions of the piezoelectric actuator 400, thus inhibiting the transfer of current from the piezoelectric element 410 to remaining portions of the piezoelectric actuator 400.

In the example shown in FIG. 4, a peripheral edge portion of the example isolation layer 430 has a sawtooth pattern, simply for purposes of discussion and illustration. The isolation layer 430 may have other shapes and/or contours based on, for example, a shape and/or contour of the piezoelectric element 410, a shape and/or contour of the diaphragm 450, associated deformation properties of the piezoelectric element 410 and/or the diaphragm 450, and other such factors.

In some examples, epoxy material disposed between the piezoelectric element 410 and the diaphragm 450 may provide for electrical isolation between the piezoelectric element 410 and the remaining portions of the piezoelectric actuator 400, alone or together with the isolation layer 430. In some examples, properties, for example, dielectric properties, of a material of the first epoxy layer 420 and/or the second epoxy layer 440 may provide for electrical isolation, and may serve as an electrical insulator between the active piezoelectric element 410 and the non-active elements of the piezoelectric actuator 400, i.e., the diaphragm 450. In some examples, a dispensing or application pattern of an epoxy layer such as the first epoxy layer 420 and/or the second epoxy layer 440 may be designed to provide for isolation of electrically active areas of the piezoelectric element 410. In the example shown in FIG. 4, the first epoxy layer 420 and the second epoxy layer 440 are deposited in a relatively large disc format, for example, corresponding to a size and/or a shape of the piezoelectric element 410. In some examples, an epoxy layer, such as, for example, the first epoxy layer 420 and/or the second epoxy layer 440, may be deposited in other types of patterns such as, for example, a ring pattern, a line pattern, a grid pattern, a sawtooth pattern, a zig zag pattern, and other such patterns that will ensure active areas of the piezoelectric element 410 will be isolated by the insulative properties of the epoxy material.

In some examples, processes associated with the application of the epoxy material may improve the effectiveness of the epoxy material as an insulator (either alone or in combination with the isolation layer 430). For example, a process in which voids, or entrapped air, or bubbles, from the epoxy material prior to application of the epoxy material may improve the uniform application of the epoxy material (for example, the first epoxy layer 420 and/or the second epoxy layer 440, and/or other epoxy layers not specifically shown in FIG. 4), and may improve the insulative characteristics of the epoxy material. In some examples, a vacuum degassing process may be used to remove voids, or entrapped air from the epoxy material prior to application. In some examples, a centrifuge process may be used to remove voids, or entrapped air from the epoxy material prior to application. In some examples, a heating process may be used to remove voids, or entrapped air from the epoxy material prior to application. In some examples, a thickness of the epoxy material (for example, the first epoxy layer 420 and/or the second epoxy layer 440, and/or other epoxy layers not specifically shown in FIG. 4) may be controlled during the application process to provide for substantially uniform application of the epoxy material, and to within a specified range of thickness that will provide the desired insulating characteristics.

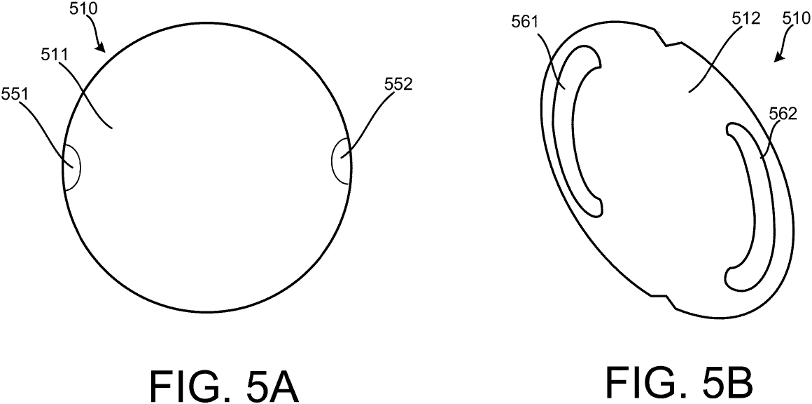

FIG. 5A is a plan view of a first side 511 of an example actuator foil 510, and FIG. 5B is a perspective view of a second side 512 of the example actuator foil 510, according to an aspect. The example actuator foil 510 shown in FIGS. 5A and 5B can be used in a piezoelectric actuator such as, for example, the piezoelectric actuator 400 shown in FIG. 4, or another piezoelectric actuator, to actuate a pump or a valve included in a fluid control system of an implantable fluid operated inflatable device, according to an aspect.

As shown in FIGS. 5A and 5B, the example actuator foil 510 includes a first electrode 551 and a second electrode 552 formed on the first side 511 of the actuator foil 510. A first recess 561 and a second recess 562 are formed in the second side 512. The second side 512 of the example actuator foil 510 may be coupled by an epoxy layer to an inactive portion of a piezoelectric actuator in which the actuator foil 510 is installed (not shown in FIGS. 5A and 5B). A position of the first electrode 551 on the first side 511 may correspond to a region covered by the first recess 561 on the second side 512. Similarly, a position of the second electrode 552 may correspond to a region covered by the second recess 562 on the second side 512. When applying epoxy to bond the second side 512 of the actuator foil 510 for bonding to another, inactive element, epoxy may also be filled in the recesses 561, 562, thus increasing a thickness of the epoxy layer in the area of the recesses 561, 562. The greater thickness of epoxy layer in the area of the recesses 561, 562 provides additional insulation and/or isolation capability in the area of the recesses 561, 562. The positioning of the electrodes 551, 552 on the first side 511 of the actuator foil 510 at positions corresponding to regions covered by the recesses 561, 562 on the second side 512 thus provides for electrical insulation and/or isolation specifically in the areas corresponding to the electrodes 551, 552.

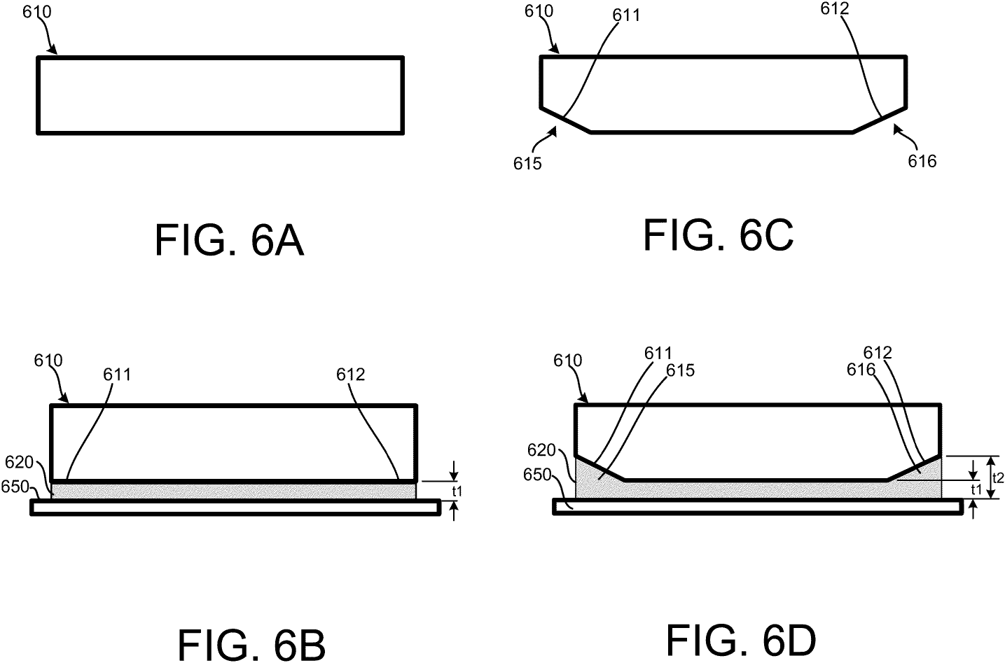

FIG. 6A schematically illustrates example piezoelectric element 610, and FIG. 6B illustrates the example piezoelectric element 610 coupled to an inactive deformable member 650, such as a diaphragm or another inactive deformable element, by an epoxy layer 620. In the example arrangement shown in FIG. 6B, electrode areas 611, 612 at which electrodes are placed to selectively actuate the piezoelectric element 610 are isolated from the inactive deformable member 650 by a thickness t1 of the epoxy layer 620.

FIG. 6C illustrates an example in which material has been removed from a first portion of the piezoelectric element 610 to form a first cutaway portion 615 corresponding to the first electrode area 611, and to form a second cutaway portion 616 corresponding to the second electrode area 612. In the example arrangement shown in FIG. 6D, the space between the piezoelectric element 610 and the inactive deformable member 650 is again filled with the epoxy layer 620. However, in the example arrangement shown in FIG. 6C, the thickness t2 of the epoxy layer 620 in the cutaway portions 615, 616 corresponding to the electrode areas 611, 612 is greater than the thickness t1 of the remaining portions of the epoxy layer 620.

The greater thickness t2 of the epoxy layer 620 in the cutaway portions 615, 616 provides an increased isolation distance at the electrode areas 611, 612, and increased levels of electrical isolation in the electrode areas 611, 612. The increased level of electrical isolation in the electrode areas 611, 612 provided by the increased thickness t2 in the cutaway portions 615, 616 may further inhibit the transfer of current into inactive portions of a piezoelectric actuator in which the piezoelectric element 610 is installed. In some examples, material properties of the material of the epoxy layer 620, and in particular material stiffness, may be matched with material properties, and in particular material stiffness of the piezoelectric element 610, to provide for coordinated deformation of the piezoelectric element 610, the epoxy layer 620, and the deformable member 650, and for adhesion across the increased thickness t2 during deformation. The example piezoelectric element 610 shown in FIG. 6D can be used in a piezoelectric actuator such as, for example, the piezoelectric actuator 400 shown in FIG. 4, or another piezoelectric actuator, to actuate a pump or a valve included in a fluid control system of an implantable fluid operated inflatable device, according to an aspect.

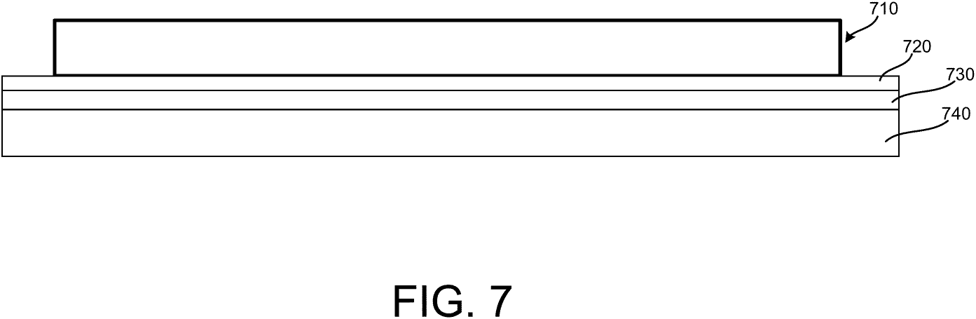

FIG. 7 schematically illustrates an example piezoelectric element 710 including a coating material having insulative properties, according to an aspect. The example piezoelectric element 710 shown in FIG. 7 can be used in a piezoelectric actuator such as, for example, the piezoelectric actuator 400 shown in FIG. 4, or another piezoelectric actuator, to actuate a pump or a valve included in a fluid control system of an implantable fluid operated inflatable device, according to an aspect.

In the example arrangement shown in FIG. 7, a coating layer 730 is deposited between the piezoelectric element 710 and an actuator foil 740. In some examples, an epoxy layer 720 may be applied between the piezoelectric element 710 and the coating layer 730. The coating layer 730 may include an insulative coating material. In some examples, the coating layer 730 may include a nano thickness layer of ceramic material. In some examples, the coating layer 730 may be applied utilizing, for example, a vapor deposition process, an atomic layer deposition (WLD) process, a parylene deposition process, and other such deposition processes. As noted above, isolation provided by the coating layer 730 may prevent voltage from leaking into fluid in fluid passageways of a pump in which the piezoelectric element is installed, which could otherwise adversely affect the patient, cause corrosion of other components of the pump, cause shorting or other malfunction of the piezoelectric actuator, and the like. Additionally, isolation of voltage in this manner inhibits transmission of voltage to the patient, thus enhancing patient safety and comfort.

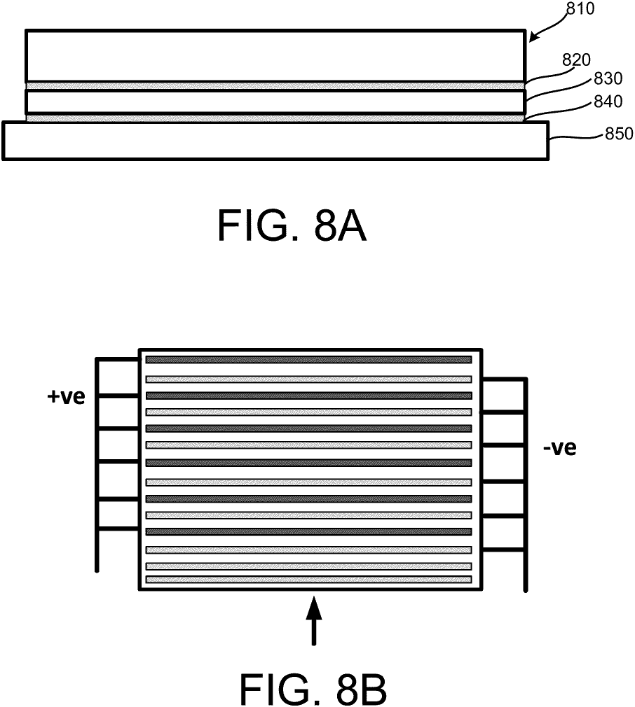

FIG. 8A schematically illustrates an example piezoelectric element 810 coupled to a ceramic layer having insulative properties, according to an aspect. FIG. 8B schematically illustrates multiple ceramic layers coupled to the example piezoelectric element The example piezoelectric element 810 shown in FIGS. 8A and 8B can be used in a piezoelectric actuator such as, for example, the piezoelectric actuator 400 shown in FIG. 4, or another piezoelectric actuator, to actuate a pump or a valve included in a fluid control system of an implantable fluid operated inflatable device, according to an aspect.

In the example arrangement shown in FIG. 8A. a ceramic layer 830 is positioned between the piezoelectric element 810 and an actuator foil 850. A material of the ceramic layer 830 may include insulative properties. In some examples, the ceramic layer 830 is joined to the piezoelectric element 810 by a first epoxy layer 820, and to the actuator foil 850 by a second epoxy layer 840. Curing of the first epoxy layer 820 and the second epoxy layer 840 may bond the ceramic layer 830 between the piezoelectric element 810 and the actuator foil 850, prior to the coupling of the piezoelectric element 810 to remaining components of a piezoelectric actuator in which the piezoelectric element 810 is to be installed. In some examples, one or more additional ceramic layers can be coupled to the piezoelectric element 810 to provide for additional isolation thickness and additional isolation. FIG. 8B illustrates a plurality of sintered isolation layers of ceramic material, that may be coupled to the piezoelectric element 810 to increase the isolation provided by the ceramic layer 830 shown in FIG. 8A. Sintering may provide for compaction of the ceramic material in the ceramic layers, providing greater isolating characteristics in a compacted form. As noted above, isolation provided by the ceramic layer may prevent voltage from leaking into fluid in fluid passageways of a pump in which the piezoelectric element is installed, which could otherwise be transmitted to the patient, adversely affecting the patient, cause corrosion of other components of the pump, cause shorting or other malfunction of the piezoelectric actuator, and the like.

In some examples, insulative materials or layers may be positioned between active portion(s) of a piezoelectric actuator, such as a piezoelectric element, and inactive portions of the piezoelectric actuator, such as a deformable member, as shown in FIGS. 4-6D. In some examples, insulative materials or layers may be positioned between the piezoelectric element and an actuator film, as shown in FIGS. 7-8B. In some examples, the insulative material may be in the form of a mesh material. In some examples, the mesh material may provide for some measure of control of a distance between elements on opposite sides of the insulative mesh material. For example, such an insulative mesh material may maintain a set minimum distance between the piezoelectric element and the actuator foil. Epoxy applied in the area of the insulative mesh material may fill openings defined in the mesh material to provide for full isolation across the surface of the layer of insulative mesh material.

In some examples, the insulative material may include microbeads of insulative material having a known size that can control the distance between elements on opposite sides of the insulative material. For example, insulative material including microbeads having a known size may maintain a minimum set distance between the piezoelectric element and the actuator foil, with epoxy deposited to provide for the bonding of the adjacent elements and the microbeads maintaining the set distance between the adjacent elements.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An implantable fluid operated inflatable device, comprising:
a fluid reservoir;
an inflatable member; and
a fluid control system configured to control fluid flow between the fluid reservoir and the inflatable member, the fluid control system including:
a housing;
fluidic architecture defining one or more fluid passageways within the housing; and
a piezoelectric actuator actuating at least one pump or at least one valve positioned in the one or more fluid passageways, the piezoelectric actuator including:
a deformable member mounted in a fluid passageway of the one or more fluid passageways defined within the housing to control a flow of fluid through the fluid passageway;
a piezoelectric element coupled to the deformable member and configured to deform in response to a voltage applied by an electronic control system of the fluid control system; and
an isolation layer positioned between the piezoelectric element and the deformable member and configured to electrically isolate the deformable member from the piezoelectric element.

2. The implantable fluid operated inflatable device of claim 1, further comprising:
a first epoxy layer coupling the isolation layer to the piezoelectric element; and
a second epoxy layer coupling the isolation layer to the deformable member.

3. The implantable fluid operated inflatable device of claim 2, wherein at least one of the first epoxy layer or the second epoxy layer is applied in a pattern, the pattern including one of a pattern corresponding to a contour of at least one of the piezoelectric element or the deformable member, a lined pattern, a mesh pattern, or a sawtooth pattern.

4. The implantable fluid operated inflatable device of claim 2, wherein a material of at least one of the first epoxy layer or the second epoxy layer is a polymer material processed prior to application to remove voids.

5. The implantable fluid operated inflatable device of claim 1, wherein an outer peripheral dimension of the isolation layer is greater than or equal to an outer peripheral dimension of the piezoelectric element.

6. The implantable fluid operated inflatable device of claim 1, wherein the isolation layer includes a mesh material or a woven material having a set thickness across the isolation layer.

7. The implantable fluid operated inflatable device of claim 1, wherein the isolation layer includes a dielectric material.

8. The implantable fluid operated inflatable device of claim 1, wherein the piezoelectric element includes:
at least one electrode on a first side of the piezoelectric element; and
at least one recess formed in a second side of the piezoelectric element, at a position corresponding to the at least one electrode.

9. The implantable fluid operated inflatable device of claim 8, wherein a contour of the at least one recess extends beyond a contour of the at least one electrode.

10. The implantable fluid operated inflatable device of claim 8, wherein the isolation layer includes an epoxy layer, and wherein a thickness of the epoxy layer at a position corresponding to the at least one recess and the at least one electrode is greater than a thickness of remaining portions of the epoxy layer.

11. The implantable fluid operated inflatable device of claim 1, wherein the piezoelectric element includes:
a first cutaway portion corresponding to a placement position of a first electrode on the piezoelectric element; and
a second cutaway portion corresponding to a placement position of a second electrode on the piezoelectric element.

12. The implantable fluid operated inflatable device of claim 11, wherein a thickness of a portion of the isolation layer corresponding to the first cutaway portion, and a thickness of a portion of the isolation layer corresponding to the second cutaway portion, is greater than a thickness of remaining portions of the isolation layer.

13. The implantable fluid operated inflatable device of claim 12, wherein the isolation layer includes an epoxy layer, and wherein a material stiffness of the epoxy layer corresponds to a material stiffness of the piezoelectric element.

14. An implantable fluid operated inflatable device, comprising:
a fluid reservoir;
an inflatable member; and
a fluid control system coupled between the fluid reservoir and the inflatable member and configured to control fluid flow between the fluid reservoir and the inflatable member, the fluid control system including:
a housing;
fluidic architecture defining one or more fluid passageways within in the housing; and
a piezoelectric actuator actuating at least one pump or at least one valve positioned in the one or more fluid passageways, the piezoelectric actuator including:
a piezoelectric element configured to deform in response to a voltage applied by an electronic control system of the fluid control system;
an actuator foil coupled to the piezoelectric element; and
an isolation layer between the piezoelectric element and the actuator foil.

15. The implantable fluid operated inflatable device of claim 14, wherein the isolation layer includes:
a coating layer deposited on one of the actuator foil or the piezoelectric element; and
an epoxy layer between the coating layer and the other of the actuator foil or the piezoelectric element.

16. The implantable fluid operated inflatable device of claim 15, wherein the coating layer includes a nano-thickness layer of a ceramic material deposited on the one of the actuator foil or the piezoelectric element.

17. The implantable fluid operated inflatable device of claim 14, wherein the isolation layer includes:
at least one ceramic layer;
a first epoxy layer bonding the at least one ceramic layer and the piezoelectric element; and
a second epoxy layer bonding the at least one ceramic layer and the actuator foil.

18. The implantable fluid operated inflatable device of claim 17, wherein an outer peripheral contour of the at least one ceramic layer is greater than or equal to a corresponding outer peripheral contour of the piezoelectric element.

19. The implantable fluid operated inflatable device of claim 14, wherein the isolation layer includes:
a plurality of microbeads positioned between the piezoelectric element and the actuator foil, wherein the plurality of microbeads are made of an insulative material and have a set size so as to maintain a set distance between the piezoelectric element and the actuator foil; and
an epoxy material applied between the piezoelectric element and the actuator foil and configured to bond the piezoelectric element, the actuator foil, and the plurality of microbeads.

20. The implantable fluid operated inflatable device of claim 14, wherein the isolation layer includes:
a mesh material positioned between the piezoelectric element and the actuator foil, wherein the mesh material is made of an insulative material and has a set thickness so as to maintain a set distance between the piezoelectric element and the actuator foil; and
an epoxy material applied between the piezoelectric element and the actuator foil, and in openings in the mesh material, and configured to bond the piezoelectric element, the actuator foil, and the mesh material.
